(19) United States
(12) Patent Application Publication
(10) Pub. No.: US 2023/0285317 A1
(43) Pub. Date: Sep. 14, 2023

(54) DILUENTS, STANDARDIZATION AGENTS AND VISCOSITY CONTROL FOR CANNABINOIDS FORMULATIONS AND METHODS THEREOF

(71) Applicant: Scientific Holdings, LLC, Monrovia, CA (US)

(72) Inventors: Bradley J. Douglass, Monrovia, CA (US); Jeffrey Charles Raber, Monrovia, CA (US)

(73) Assignee: Scientific Holdings, LLC, Monrovia, CA (US)

(21) Appl. No.: 18/120,975

(22) Filed: Mar. 13, 2023

Related U.S. Application Data
(60) Provisional application No. 63/319,632, filed on Mar. 14, 2022.

(51) Int. Cl. A61K 31/05; A61K 31/60; A61K 47/10; G01N 33/15

(52) U.S. Cl. CPC A61K 31/05; A61K 31/60; A61K 47/10; G01N 33/15

(57) ABSTRACT

The formulation provides a cannabinoid diluent and viscosity standardization formulation that uses CBC and bisabolol that are found naturally occurring in Cannabaceae as solvating, diluting, and/or viscosity standardizing agents. A method to identify individual compounds and mixtures of compounds to create optimized diluents for a range of cannabinoids utilizing Hansen Solubility Parameters (HSP) principles is also provided. The method utilizes HSP to determine the best individual and combinations of components to effectively solubilize and standardize cannabinoid formulations intended for vaporization or aerosolization. The composition of matter includes multi-component formulations that solubilize and standardize the viscosity of cannabinoids.

This patent application claims priority to U.S. Provisional Patent Application 63/319,632 filed on Mar. 14, 2022, which is incorporated by reference herein in its entirety.

### FIELD

The formulation generally relates to vaping, and more specifically, to thermally-stable formulations and combinations of components that effectively solubilize and standardize the viscosity of cannabinoids and cannabis extracts intended for vaporization or aerosolization.

### BACKGROUND

From mid-2019 onwards an outbreak of vaping-related respiratory illness was observed in the United States. This was recognized as a new disease entity, designated ‘e-cigarette, or vaping, product use—associated lung injury (EVALI). Work was conducted to link product-sample analyses with patient case numbers. As of Feb. 12, 2020, 93 EVALI case-associated samples had been analyzed. Of these, 73% contained tetrahydrocannabinol (THC), 81% included vitamin E acetate (VEA) as a diluent, 32% contained aliphatic esters (for example, triglycerides or medium-chain triglycerides, MCT), and 9% contained polyethylene glycol (PEG) as diluent. Additional samples provided by those afflicted with EVALI further strengthened the link to VEA.

VEA had been commercialized by incompetent and self-promoting salesmen in the illicit cannabis market as an ingredient that could both dilute the cannabinoid content and standardize the viscosity or thickness of a vape formulation while maintaining a clear, colorless, and visually appealing product. The pitch was that VEA, a substance that is generally recognized as safe for food-use in the United States could be used as an inexpensive additive to “cut” the high-cost tetrahydrocannabinol (THC), cannabidiol (CBD), and/or cannabinoid-enriched cannabis extract. Generally, oil that has a higher percentage of cannabinoids is thicker and consumers of illicit vape products commonly used this property to assess quality or purity in the absence of test results. So unwitting consumers purchased vape products containing 10-90% VEA believing they were getting more cannabinoids than the product actually contained and often without being aware that VEA had been incorporated as an excipient. EVALI was largely avoided in the regulated adult-use cannabis marketplaces since testing and labeling requirements prevented such VEA usage. To address the key issue of whether VEA was simply a marker or a significant mediator of EVALI, Wu and O'Shea reported in PNAS that VEA reacts when aerosolized at certain elevated temperatures to produce highly toxic ketene gas.

Meanwhile, public health researchers were uncovering a strong correlation between the risk of adolescent psychosis and high-THC or “high-potency” cannabis via epidemiological research and anecdotal evidence. Despite a demonstrable lack of a causative link between “high-potency” cannabis and childhood psychosis, even amongst a subset of adolescents, or even a correlative link between cannabis concentrates or vape products and childhood psychosis, legislation was proposed to limit the THC potency of vape products in a number of U.S. states based upon these findings. When proposing or passing such potency-limiting legislation, little thought was given to what the limited THC would be replaced with and thus not considering that there were few, if any, known “diluent” options that would be safer than the THC that would be replaced. This is in stark contrast to how alcohol products are largely diluted with water, one of the most benign substances to ingest. There is no such analogous substance to water with respect to inhalation that is also a liquid at room-temperature.

In addition, researchers began to report that the proper functioning of vape and aerosol devices required that the oil to be vaped had a viscosity upper limit or the material would not travel quickly enough (“wick”) onto the heating element. Without such viscosity control, the heating element designs of these devices would allow runaway heating and temperature spikes. This is the very behavior that could lead to VEA converting into ketene gas considering that under proper conditions most vaporization or aerosolization devices will not reach the high temperatures required to catalyze the degradation chemistry of VEA to ketene.

In addition, it became evident that the proper functioning of vape and aerosol devices required that the oil to be vaped remain above a threshold viscosity lower limit to eliminate the potential for the material to unintentionally leave the oil storage reservoir (cf. “leak”) when the vape or aerosol device was not in use. This would lead to oil exiting the cartridge and soiling the user's pocket, bag, or keepsafe. This then led to unhappy consumers or, worse yet, many failed and unsalvageable cartridges between the manufacturing facility and the store shelf. In general, 35 degrees Celsius (95 degrees Fahrenheit) is a good test case for determining the lower limit of viscosity for vape device applications because it represents the upper limit of ambient temperature in most temperate regions of the globe.

It is apparent that a need exists to create formulations and methods directed toward the safe and optimized standardization, solubility and viscosity control (a.k.a. “diluents”) for a range of individual cannabinoids, mixtures of cannabinoids and cannabis extracts.

### SUMMARY

This formulation provides thermally-stable cannabinoid diluent and standardization formulations using compounds found naturally occurring in the botanical family Cannabaceae.

According to a first aspect, cannabinoid diluent and viscosity standardization formulations are provided that use cannabichromene (CBC) and bisabolol as solvating, diluting, and/or viscosity standardizing agents. This is particularly beneficial because both CBC and bisabolol are known to have benign, or even salubrious, properties with respect to tissues of the lung and nasopharynx. According to another aspect, a method to identify individual compounds and mixtures of compounds to create optimized diluents for a range of cannabinoids utilizing Hansen Solubility Parameters (HSP) principles is provided and cross-referenced with viscosity data on the optimized mixtures. The method utilizes HSP to determine desirable individual, and combinations of, components to effectively solubilize and standardize cannabinoid formulations intended for vaporization or aerosolization by evaluating non-polar (dispersion), permanent dipole (polar) and hydrogen-bonding interactions, then cross-referencing the results with experimentally obtained viscosity data of the mixtures to confirm an acceptable range.

There does not exist any reliable means to predict the viscosity of liquid mixtures (beyond perhaps very non-polar mixtures, such as hydrocarbons), so viscosity data must be generated experimentally to obtain any sort of reliability. The composition of matter aspect of this formulation includes single and multi-component formulations that solubilize and standardize cannabinoids. HSP evaluation determined that CBC and bisabolol, both of which are found naturally-occurring in the botanical family Cannabaceae, possessed unexpected solubility properties amenable to dissolving a variety of cannabinoids. These include neutral cannabinoids, such as tetrahydrocannabinol (THC), cannabidiol (CBD), and cannabigerol (CBG), as well as acidic cannabinoids such as tetrahydrocannabinolic acid (THCA), cannabidiolic acid (CBDA), and cannabigerolic acid (CBGA).

According to an embodiment, CBC is used to create a flowable and stable oil out of solid compounds CBD, CBG and CBDA up to concentrations of 80% CBD, 40% CBG and 20% CBDA. According to another embodiment, bisabolol is used to create a flowable and stable oil containing greater than 60% CBG. Mixed-in various relative concentrations to each other, CBC and bisabolol mixtures can be tailored to achieve a diluent composition to address practically any combination of cannabinoids and to extend across a wide viscosity range. For example, a flowable oil consisting of 30% CBD and 40% CBG can be achieved using a range of CBC and bisabolol to standardize the viscosity of the final oil.

### DETAILED DESCRIPTION

The terminology used herein is for the purpose of describing particular embodiments only and is not intended to be limiting of the formulation. As used herein, the term “and/or” includes any and all combinations of one or more of the associated listed items. As used herein, the singular forms “a,” “an,” and “the” are intended to include the plural forms as well as the singular forms, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof.

Unless otherwise defined, all terms (including technical and scientific terms) used herein have the same meaning as commonly understood by one having ordinary skill in the art to which this formulation belongs. It will be further understood that terms, such as those defined in commonly used dictionaries, should be interpreted as having a meaning that is consistent with their meaning in the context of the relevant art and the present disclosure and will not be interpreted in an idealized or overly formal sense unless expressly so defined herein.

In describing the formulation, it will be understood that a number of techniques and steps are disclosed. Each of these has individual benefit and each can also be used in conjunction with one or more, or in some cases all, of the other disclosed techniques. Accordingly, for the sake of clarity, this description will refrain from repeating every possible combination of the individual steps in an unnecessary fashion. Nevertheless, the specification and claims should be read with the understanding that such combinations are entirely within the scope of the formulation and the claims. In the following description, for purposes of explanation, numerous specific details are set forth in order to provide a thorough understanding of the formulation. It will be evident, however, to one skilled in the art that the formulation may be practiced without these specific details. The present disclosure is to be considered as an exemplification of the formulation and is not intended to limit the formulation to the specific embodiments illustrated by the description below.

According to the formulation cannabichromene (CBC), bisabolol and other compounds found naturally occurring in botanical family Cannabaceae have solubility properties amenable to dissolving a variety of cannabinoids. These include neutral cannabinoids, such as THC, CBD, and CBG, as well as acidic cannabinoids such as THCA, CBDA, and CBGA. The combined use of cannabichromene (CBC) and bisabolol in vaporizable compositions comprising cannabinoids provided several beneficial effects. The formulation efficiently solves an issue of poor reproducibility commonly encountered with vape and aerosolization devices that negatively affects the ability to provide consistent and reproducible amounts of cannabinoid formulations.

The weight percent of CBC and bisabolol runs the entire spectrum of 0-100% CBC and 0-100% bisabolol depending on the solubility characteristics required of the cannabinoid formulation and the target viscosity of the finished formulation. The weight percent of CBC and bisabolol together ranges from 5% to 95% depending on the targeted cannabinoid concentration (cf. potency) of the finished formulation, the desired quantity of other additives, such as flavor compounds, and the target viscosity of the finished formulation.

The concentrations of CBC and bisabolol are typically greater than what can be produced by any other known diluent ingredients. In addition, both CBC and bisabolol possess chemical structures that are not heat-labile. This means that even when heated to unexpectedly high temperatures, as can occur in improperly designed vape or aerosolization devices or in the improper pairing of a non-optimized power source with the atomization or aerosolization device, the CBC or bisabolol do not degrade into other compounds that could produce toxicological insults. This is particularly beneficial because both CBC and bisabolol are known to have benign, or even salubrious, properties with respect to tissues of the lung and nasopharynx and so possess an attractive toxicological profile in and of themselves.

Table 5 shows the viscosity readings at 35 degrees Celsius for mixtures composed of cannabidiol (CBD) at 10, 40 and 70 percent by mass and CBC and/or bisabolol ranging from all CBC to all bisabolol and various and symmetrical ratios of CBC to bisabolol in between. One example is also provided of the effects of adding 10% by mass of a composite flavor additive composed of terpenes and terpenoids

Table 6 shows the viscosity readings at 35 degrees Celsius for mixtures composed of cannabigerol (CBG) at 10, 40 and 70 percent by mass and CBC and/or bisabolol ranging from all CBC to all bisabolol and various and symmetrical ratios of CBC to bisabolol in between. One example is also provided of the unexpected effects of adding 10% by mass of a composite flavor additive composed of terpenes and terpenoids that led to stabilizing the viscosity of the formulation when the viscosity did not stabilize in the absence of the flavor additive.

In an example embodiment, the solubility of a number of solid cannabinoid isolates in concentrations of 10-80 percent by mass were evaluated in CBC, a viscous oil at room-temperature. Solubility is assessed at 25 degrees (Celsius), after heating to 35 degrees (Celsius), after heating to 70 degrees (Celsius), and after heating to 100 degrees (Celsius). The solubility of cannabinoids with CBC is then observed at room temperature at various time points after formulation to determine long-term solubility and oil stability. If full dissolution does not occur within 10-15 minutes of agitation at 25° C., the sample is progressively heated until it does at one of the subsequent three preset temperature thresholds or until it fails to dissolve at 100° C.

Experimental Equipment:
1) Glass Vials, 2 ml
2) Balance
3) Spatulas
4) Hot plates, 3×
5) CBD (5 g), CBG (5 g), CBDA (5 g)

### EXAMPLE 1: SOLUBILITY OF CBD AT ROOM TEMPERATURE, AT 35 DEGREES (CELSIUS), AT 70 DEGREES (CELSIUS), AT 100 DEGREES (CELSIUS)

TABLE 1

| S. | Canna- | Cannabi- | Cannabi- | CBC | Tare | Cannabi- | CBC |
|---|---|---|---|---|---|---|---|
| No. | binoid | noid (%) | noid (mg) | (mg) | Wt. | noid (g) | (g) |
| 1 | CBD | 10% | 100 | 900 | 4.674 | 0.100 | 0.905 |
| 2 | CBD | 20% | 200 | 800 | 4.645 | 0.200 | 0.806 |
| 3 | CBD | 30% | 300 | 700 | 4.619 | 0.300 | 0.700 |
| 4 | CBD | 40% | 400 | 600 | 4.634 | 0.403 | 0.605 |
| 5 | CBD | 50% | 500 | 500 | 4.646 | 0.502 | 0.503 |
| 6 | CBD | 60% | 600 | 400 | 4.624 | 0.600 | 0.403 |
| 7 | CBD | 70% | 700 | 300 | 4.647 | 0.702 | 0.304 |
| 8 | CBD | 80% | 800 | 200 | 4.641 | 0.803 | 0.200 |

CBC was added to the serially-numbered glass vials containing CBD at the various concentrations shown in Table 1. The samples were then heated at an appropriate temperature for 15 total minutes in a water bath and then if any of the CBD remained solid, the vial would be heated for 15 minutes at the next temperature level. A glass thermometer was used to observe and record the temperature. All eight vials corresponding to the eight concentrations of CBD in CBC became a homogeneous mass upon 6-8 minutes heating at 70 degrees (Celsius). Vials 1-4 exhibited zero crystallization and retained a viscosity similar to corn syrup [viscosity range: 2,000-3,000 centipoise (cP)] after being held at room temperature for 1 and 4 week time points. Vials 5 and 6, representing 50% and 60% CBD in CBC respectively, also exhibited zero crystallization and retained a viscosity similar to molasses [viscosity range: 5,000-10,000 cP)] after being held at room temperature for 1 and 4 week time points. Vials 7 and 8, representing 70% and 80% CBD in CBC respectively, also exhibited zero crystallization and retained a viscosity similar to THC distillate throughout the 1 and 4 week time course at room temperature (i.e., 25° C.)

TABLE 2

| S. | Canna- | Cannabi- | Cannabi- | CBC | Tare | Cannabi- | CBC |
|---|---|---|---|---|---|---|---|
| No. | binoid | noid (%) | noid (mg) | (mg) | Wt. | noid (g) | (g) |
| 9 | CBG | 10% | 100 | 900 | 4.635 | 0.101 | 0.903 |
| 10 | CBG | 20% | 200 | 800 | 4.642 | 0.200 | 0.806 |
| 11 | CBG | 30% | 300 | 700 | 4.649 | 0.303 | 0.704 |
| 12 | CBG | 40% | 400 | 600 | 4.661 | 0.402 | 0.600 |
| 13 | CBG | 50% | 500 | 500 | 4.669 | 0.502 | 0.504 |
| 14 | CBG | 60% | 600 | 400 | 4.641 | 0.600 | 0.402 |
| 15 | CBG | 70% | 700 | 300 | 4.647 | 0.702 | 0.304 |
| 16 | CBG | 80% | 800 | 200 | 4.656 | 0.800 | 0.203 |

CBC was added to the serially-numbered glass vials containing CBG at the various concentrations shown in Table 2. The samples were then heated at an appropriate temperature for 15 total minutes in a water bath and then if any of the CBG remained solid the vial would be heated for 15 minutes at the next temperature level. A glass thermometer was used to observe and record the temperature. Vial 9 containing 10% CBG and Vial 10 containing 20% CBG has zero solid material remaining after 15 minutes at 35° C. Vials 11-16, containing 30% CBG to 80% CBG at 10 percent weight increments, had some remaining solids after 15 minutes at 30 degrees (Celsius), but none had any visible solids remaining after 4 minutes at 70° C. The viscosity of the solution containing 50% CBG (Vial 13) and 60% CBG (Vial 14) increased upon standing for 35 to 40 minutes at room temperature. Some crystallization of the solution containing 70% CBG (Vial 15) occurs upon standing at room temperature for 5 to 8 minutes. Significant crystallization of Vial 16 (80% CBG) occurs after 5 to 8 minutes at room temperature.

TABLE 3

| S. | Canna- | Cannabi- | Cannabi- | CBC | Tare | Cannabi- | CBC |
|---|---|---|---|---|---|---|---|
| No. | binoid | noid (%) | noid (mg) | (mg) | Wt. | noid (g) | (g) |
| 17 | CBDA | 10% | 100 | 900 | 4.637 | 0.103 | 0.900 |
| 18 | CBDA | 20% | 200 | 800 | 4.680 | 0.200 | 0.806 |
| 19 | CBDA | 30% | 300 | 700 | 4.667 | 0.300 | 0.704 |
| 20 | CBDA | 40% | 400 | 600 | 4.650 | 0.401 | 0.605 |
| 21 | CBDA | 50% | 500 | 500 | 4.654 | 0.502 | 0.500 |
| 22 | CBDA | 60% | 600 | 400 | 4.640 | 0.602 | 0.401 |
| 23 | CBDA | 70% | 700 | 300 | 4.641 | 0.702 | 0.306 |
| 24 | CBDA | 80% | 800 | 200 | 4.654 | 0.802 | 0.205 |

CBC was added to the serially-numbered glass vials containing CBDA at the various concentrations shown in Table 3. The samples were then heated at an appropriate temperature for 15 total minutes in a water bath and then if any of the CBDA remained solid the vial would be heated for 15 minutes at the next temperature level. A glass thermometer was used to observe and record the temperature. The sample containing 10% CBDA (Vial 17) and 20% CBDA (Vial 18) had solids remaining after 15 minutes at 30° C. and after 15 minutes at 70° C. But no solids were evident after 4 minutes at 100 degrees (Celsius). The samples containing 30% CBDA (Vial 18) and 40% CBDA (Vial 19) had solids visibly remaining after 15 minutes at 30° C. as well as after 15 minutes at 70° C. Both Vials 18 and 19, however, had no visible solids remaining after 5 minutes at 100° C. Vials 20 through 24, containing 50% to 80% CBDA respectively in 10 weight percent increments, were homogenous liquids after 7 minutes at 100° C. The samples containing 30% CBDA to 60% CBDA became very viscous but remained liquid after 20 minutes at room temperature. The samples containing 70% and 80% CBDA turned into a gel after 20 minutes at room temperature.

After day 1, the samples containing 10% to 80% CBD appeared more viscous but remained homogeneous oils. The samples containing 10% to 30% CBG appeared more viscous but remained oils, while the solution containing 40% CBG showed a minimal amount of crystallization. The samples containing 50% to 80% CBG exhibited various amounts of crystallization. The samples containing 10% to 40% CBDA appear viscous but remain homogeneous oils, while the sample containing 50% CBDA has become very viscous. The samples containing 60% to 80% CBDA turned into solid gels.

After day 8, samples containing 10% to 40% CBD displayed no crystallization and the solution appeared viscous (like corn syrup) and samples containing 50% and 60% CBD also exhibited no crystallization but had attained a very viscous consistency (like molasses). Samples containing 70% and 80% CBD showed no crystallization and appeared even more viscous almost to the viscosity level of THC distillate (ca. 85% THC). Samples containing 10% to 30% CBG showed no crystallization, but molasses-like consistency. The 40% CBG sample in CBC showed partial crystallization, while the sample containing 50% CBG in CBC showed crystallization of that visually extended to the height of the sample level inside the vial. The sample containing 60% to 80% CBG in CBC all appeared fully crystallized. The samples containing 10% to 30% CBDA in CBD did not exhibit any crystallization after eight days standing at 25° C., but the homogenous liquids appeared very viscous. The sample containing 40% CBDA showed no crystallization and appeared very viscous almost to the level of THC distillate (ca. 85% THC). Samples containing 50% to 80% CBDA remained solid gels.

After one month, samples containing 10% to 40% CBD exhibited no crystallization and the solution appeared viscous like corn syrup. Samples containing 50% and 60% CBD showed no crystallization and appeared very viscous (similar to molasses). Samples containing 70% and 80% CBD exhibited no crystallization and also appeared even more viscous similar to THC distillate (ca. 85% THC). The samples containing 10% to 30% CBG in CBG displayed no visible solidification, but had become very viscous. The samples containing 40% and 50% CBG exhibited partial crystallization. The samples containing 60% to 80% CBG in looked fully crystalized. The samples containing 10% and 20% CBDA in CBC exhibited no crystallization and the solution appeared viscous like molasses. Samples containing 30% and 40% CBDA also showed no solidification but had become even more viscous. Samples containing 50% to 80% CBDA appeared about as viscous as 80% THC distillate (5,000-9,000 cP@40° C.) but exhibited no visible solidification.

### EXAMPLE 2: SOLUBILITY OF 70% CBD, CBG AND CBDA IN CBC AND BISABOLOL

TABLE 4

| S. | | Cannabinoid | CBC | Bisabolol | Actual | Mass % |
|---|---|---|---|---|---|---|
| No. | Cannabinoid | (mg) | (mg) | (mg) | AB (g) | of AB |
| 1. | CBD | 700 | 300 | 111.111 | 0.114 | 10.000 |
| 2. | CBG | 700 | 300 | 111.111 | 0.113 | 10.000 |
| 3. | CBDA | 700 | 300 | 111.111 | 0.113 | 10.000 |

The sample comprising 700 mg CBD showed no crystallization and the solution appeared viscous like corn syrup after adding bisabolol at 10% by mass. The sample comprising 700 mg CBG showed no crystallization and the solution appeared viscous like corn syrup after adding bisabolol at 10% by mass and heating the sample to around 85° C. The sample comprising 700 CBDA showed no crystallization and the solution appeared viscous like corn syrup after adding bisabolol at 10% by mass. After one day, the solution comprising 700 mg CBD showed no crystallization and the solution appeared viscous like molasses. The solution comprising 700 mg CBG showed partial crystallization. The solution comprising 700 mg CBDA appeared viscous as 85% THC distillate 80% THC distillate (5,000-9,000 cP@40° C.) without any crystallization.

### EXAMPLE 3: SOLUBILITY OF CBG IN BISABOLOL

TABLE 5

| S. | | Cannabinoid | Cannabinoid | Bisabolol | CBG | Bisabolol | Dissolution |
|---|---|---|---|---|---|---|---|
| No. | Cannabinoid | (%) | (mg) | (mg) | (g) | (g) | Temp (° C.) |
| 1. | CBG | 10% | 100 | 900 | 0.102 | 0.902 | 35 |
| 2. | CBG | 20% | 200 | 800 | 0.201 | 0.802 | 70 |
| 3. | CBG | 30% | 300 | 700 | 0.302 | 0.702 | 70 |
| 4. | CBG | 40% | 400 | 600 | 0.402 | 0.604 | 70 |
| 5. | CBG | 50% | 500 | 500 | 0.501 | 0.501 | 70 |
| 6. | CBG | 60% | 600 | 400 | 0.601 | 0.403 | 70 |
| 7. | CBG | 70% | 700 | 300 | 0.702 | 0.305 | 70 |
| 8. | CBG | 80% | 800 | 200 | 0.402 | 0.103 | 70 |

At room temperature, CBG almost went fully into bisabolol, and a few crystals were seen in the solution containing 10% CBG. More crystals were seen in the bottom of the vial containing 20% CBG compared to 10% CBG. Even more crystals were seen in the bottom of vial containing 30% CBG compared to that containing 20% CBG. More crystals still were seen in the bottom of the vial containing 40% CBG compared to 30% CBG. Many crystals were seen in the bottom of the vial containing 50% CBG. In vials containing 60% CBG, the majority of CBG was visibly solid. In the vial containing 70% CBG, much solid CBG was evident. In the vial containing 80% CBG, the solid CBG had almost completely absorbed the liquid bisabolol without any visible change of state.

After fifteen (15) minutes at 35° C., the entirety of the CBG was dissolved in the sample containing 10% CBG. Whereas only scant amounts of solid were visible in the sample containing 20% CBG. Roughly 10% of the original amount of CBG seemed to remain crystalline in the sample containing 30% CBG. Approximately 20% the original amount of CBG remained crystalline in the sample containing 40% CBG. Approximately 30% of the original amount of CBG remained solid in the sample containing 50% CBG and approximately 40% of the original mass of CBG remained solid in the solution containing 60% CBG. A significant quantity of solids/powder was evident in the samples containing 70% and 80% CBG.

After fifteen (15) minutes at 70° C., the samples containing 10% and 20% CBG became homogenous oils and remained so after allowing them to cool to room temperature for approximately 2 hours. Similarly, after heating the samples containing 30% and 40% CBG to 70 ° C. and holding for 15 minutes, then letting them cool to room temperature for approximately 2 hours, resulted in no observed crystallization. These samples had a viscosity akin to cough syrup [viscosity range: 50-100 cP at 25° C]. After heating the samples containing 50%, 60% and 70% CBG in bisabolol at 70 degrees (Celsius) for fifteen minutes and allowing them to cool to room temperature for approximately 2 hours, no crystallization was observed with the samples all exhibiting a viscosity akin to corn syrup. After heating the solution containing 80% CBG at 70 degrees (Celsius) and letting it cool to room temperature for approximately 2 hours, partial crystallization was seen in the bottom of the vial.

After 24 hours, the samples containing 10% CBG and 20% CBG showed no crystallization and the samples exhibited a viscosity similar to neat bisabolol. The samples containing 30% CBG and 40% CBG showed no crystallization and the solutions had a viscosity similar to cough syrup. The samples containing 50% CBG and 60% CBG showed no crystallization while exhibiting viscosities similar to corn syrup. The sample containing 70% CBG showed partial crystallization while the solution containing 80% CBG showed almost full crystallization.

In an example embodiment, the viscosity of two solid cannabinoid isolates (CBD and CBG) in concentrations of 10-70 percent by mass were evaluated in CBC, a viscous oil at room-temperature, and bisabolol, an oil at room-temperature. Viscosity was assessed at 35 degrees (Celsius) using a rotary viscometer with internal sample heating chamber.

Experimental Equipment:
1) Syringes, 5 ml
2) Balance
3) CBD (ca. 56 g) and CBG (ca. 56 g)
4) CBC (ca. 34 g) and bisabolol (ca. 34 g)
5) Flavor additives [i.e. Jack Herer terpene blend] (500 mg)

### EXAMPLE 4: VISCOSITY OF CBD IN CBC AND/OR BISABOLOL

TABLE 6

| | | | | Flavor | Measured |
|---|---|---|---|---|---|
| | | | | Additives | Viscosity |
| S.No | CBD (%) | CBC (%) | Bisabolol (%) | (mg) | @35 C. (cP) |
| 1 | 10% | 90% | 0% | 0 | 440.7 |
| 2 | 10% | 80% | 10% | 0 | 386.4 |
| 3 | 10% | 68% | 23% | 0 | 313.2 |
| 4 | 10% | 45% | 45% | 0 | 205.8 |
| 5 | 10% | 23% | 68% | 0 | 107.4 |
| 6 | 10% | 10% | 80% | 0 | 75.3 |
| 7 | 10% | 0% | 90% | 0 | 53.7 |
| 8 | 40% | 60% | 0% | 0 | 984 |
| 9 | 40% | 45% | 15% | 0 | 819 |
| 10 | 40% | 45% | 15% | 0 | 811 |
| 11 | 40% | 25% | 35% | 0 | 606 |
| 12 | 40% | 15% | 45% | 0 | 479 |
| 13 | 40% | 5% | 55% | 0 | 396 |
| 14 | 40% | 0% | 60% | 0 | 372 |
| 15 | 70% | 30% | 0% | 0 | 2670 |
| 16 | 70% | 18% | 12% | 0 | 2220 |
| 17 | 70% | 15% | 15% | 0 | 2280 |
| 18 | 70% | 10% | 20% | 0 | 2340 |
| 19 | 70% | 5% | 25% | 0 | 2330 |
| 20 | 70% | 2% | 28% | 0 | 2190 |
| 21 | 70% | 0% | 30% | 0 | 2100 |
| 22 | 70% | 5% | 25% | 750 | 372 |

In Example 4 (Table 6) the viscosity readings as measured at 35 degrees Celsius were directly related to the mass percent of CBD. The data also demonstrate that using only CBC as viscosity standardization agent leads to higher viscosity and only bisabolol as viscosity standardization agent leads to lower viscosity with the viscosity able to be tailored using the various ratios of CBC to bisabolol. Also, the addition of the flavor additive significantly decreased the viscosity of a 70 percent by mass formulation of CBD. Based upon the stability of the viscosity readings, the solubility (and crystallization avoidance) as predicted by HSP parameters was corroborated in practice for this system of CBD plus CBC and bisabolol.

### EXAMPLE 5: VISCOSITY OF CBG IN CBC AND/OR BISABOLOL

TABLE 7

| | | | | Flavor | Measured |
|---|---|---|---|---|---|
| | | | | Additives | Viscosity |
| S.No | CBG (%) | CBC (%) | Bisabolol (%) | (mg) | @35 C. (cP) |
| 1 | 10% | 90% | 0% | 0 | 372 |
| 2 | 10% | 80% | 10% | 0 | 324 |
| 3 | 10% | 68% | 23% | 0 | 253 |
| 4 | 10% | 45% | 45% | 0 | 179 |
| 5 | 10% | 23% | 68% | 0 | 104.2 |
| 6 | 10% | 10% | 80% | 0 | 70 |
| 7 | 10% | 0% | 90% | 0 | 54.5 |
| 8 | 40% | 60% | 0% | 0 | 288 |
| 9 | 40% | 45% | 15% | 0 | 262 |
| 10 | 40% | 45% | 15% | 0 | 258.1 |
| 11 | 40% | 25% | 35% | 0 | 210 |
| 12 | 40% | 15% | 45% | 0 | 126.6 |
| 13 | 40% | 5% | 55% | 0 | 150 |
| 14 | 40% | 0% | 60% | 0 | 128.6 |
| 15 | 70% | 30% | 0% | 0 | n/a |
| 16 | 70% | 18% | 12% | 0 | n/a |
| 17 | 70% | 15% | 15% | 0 | n/a |
| 18 | 70% | 10% | 20% | 0 | n/a |
| 19 | 70% | 5% | 25% | 0 | n/a |
| 20 | 70% | 2% | 28% | 0 | n/a |
| 21 | 70% | 0% | 30% | 0 | n/a |
| 22 | 70% | 5% | 25% | 750 | 94.5 |

In Example 5 (Table 7) the viscosity readings as measured at 35 degrees Celsius were directly related to the mass percent of CBG. The data also demonstrate that using only CBC as viscosity standardization agent leads to higher viscosity and using only bisabolol as viscosity standardization agent leads to lower viscosity with the viscosity able to be tailored using the various experimental ratios of CBC to bisabolol. Also, the addition of the flavor additive enabled the stabilization of viscosity readings for a 70% by mass CBG formulation that would not stabilize without. This can be seen in sample #s 15-21 where the lack of viscosity stabilization is denoted by the “n/a” in the Measured Viscosity column. Based upon the stability of the viscosity readings for 10% and 40% formulations of CBG, the solubility (and crystallization avoidance) as predicted by HSP parameters was corroborated in practice for this system of CBD plus CBC and bisabolol. Furthermore, it was observed that flavor additives could stabilize the viscosity of 70% CBG formulations and this indicated a positive impact of overall solubility for CBG in this composite formulation.

While the formulation has been described in terms of particular embodiments and applications, in both summarized and detailed forms, it is not intended that these descriptions in any way limit the scope of any such embodiments and applications, and it will be understood that many substitutions, changes and variations in the described embodiments, applications and details of the method and system illustrated herein and of their operation can be made by those skilled in the art without departing from the spirit of this formulation.

What is claimed is:

1. A thermally-stable and vaporizable or aerosolizable formulation comprising:
at least one cannabinoid, wherein the cannabinoid is selected from cannabidiol (CBD), cannabigerol (CBG), cannabidiolic acid (CBDA) or a combination thereof; and
at least one solubility and viscosity standardization agent, wherein the at least one standardization agent is selected from cannabichromene (CBC), bisabolol or a combination thereof

2. The formulation according to claim 1, wherein the weight percent of the at least one solubility and viscosity standardization agent ranges from 5% to 95%.

3. The formulation according to claim 2, wherein the mass ratio of CBC to bisabolol is between about 1:3 and 1:10.

4. The formulation according to claim 1, wherein the formulation is comprised of about 10 to 80 percent by mass of the at least one cannabinoid.

5. The formulation according to claim 1, wherein the formulation is comprised of about 20 to 90 percent by mass of the at least one viscosity and solubility standardization agent.

6. The formulation according to claim 1 further comprising flavor additives.

7. The formulation according to claim 1 that maintains a viscosity of between 50 and 10,000 centipoise (cP) at 35 degrees Celsius.

8. The formulation according to claim 1 in which the at least one standardization agent does not decompose at transient temperatures below 250 degrees Celsius.

9. The formulation according to claim 1 in which the at least one standardization agent does not decompose at transient temperatures below 500 degrees Celsius.

10. The formulation according to claim 1 wherein the at least one standardization agent does not decompose at transient temperatures below 750 degrees Celsius.

11. The formulation according to claim 1 wherein the at least one standardization agent does not present an inhalation hazard greater than cannabis extract.

12. The formulation according to claim 1 wherein which the at least one standardization agent helps to protect against inhalation hazards caused by cannabis extract.

13. The formulation according to claim 1 wherein the at least one standardization agent helps to protect against inhalation hazards caused by cannabinoids.

14. A method of identifying viscosity and solubility standardization agents and mixtures thereof comprising:
a. Assessing the compatibility of nonpolar, polar and hydrogen-bonding intermolecular interactions between proposed standardization agents and a least one cannabinoid or cannabis extract sought to be solubilized and,
b. Assessing the acceptability of the viscosity for the resulting formulation according to the vaporization or aerosolization device design parameters.

15. The method of claim 14 wherein the assessing of the acceptability of the viscosity is done using a viscometer at 35 degrees Celsius.

16. The method of claim 14 wherein the assessing of the acceptability of the viscosity is done by comparing the relative viscosity of the formulation with the viscosity of known substances.

17. The method of creating viscosity and solubility standardization agent(s) consisting of compounds found naturally-occurring in the botanical family Cannabaceae.

18. A method of modulating the viscosity and solubility of cannabinoid formulations comprised of at least one cannabinoid that is not CBC, with a flavor additive.

19. The method of claim 18 wherein the flavor additive is a mixture of compounds that are all found naturally-occurring in the botanical family Cannabaceae.

20. The method of claim 18 wherein the flavor additive is a mixture of compounds that are all found naturally-occurring in the botanical species Cannabis sativa.
